(19) United States
(12) Patent Application Publication
(10) Pub. No.: US 2023/0083766 A1
(43) Pub. Date: Mar. 16, 2023

(54) DIAGNOSTIC REAGENT FOR QUANTITATIVE DETERMINATION OF PROCALCITONIN IN A SAMPLE

(71) Applicant: DiaSys Diagnostic Systems GmbH, Holzheim (DE)

(72) Inventors: Matthias GRIMMLER, Elz (DE); Thomas MASETTO, Diez (DE)

(73) Assignee: DiaSys Diagnostic Systems GmbH, Holzheim (DE)

(21) Appl. No.: 17/795,513

(22) PCT Filed: Jan. 25, 2021
(86) PCT No.: PCT/EP2021/051577

§ 371 Date: Jul. 26, 2022

(30) Foreign Application Priority Data
Jan. 28, 2020 (DE) 10 2020 102 038.4

(51) Int. Cl. G01N 33/68; G01N 33/74

(52) U.S. Cl. CPC G01N 33/6854; G01N 33/74; G01N 2333/585

(57) ABSTRACT

A diagnostic reagent that is suitable for turbidimetric analysis with a simple photometer and has high sensitivity for quantitative determination of procalcitonin in a sample is provided. The reagent is an aqueous suspension of polymer particles with antibodies against procalcitonin covalently bound to said polymer particles, in which no or only an extremely slight tendency to agglutination/sedimentation is detectable even after longer standing times and the specific reactivity of the particles remains largely unchanged. The suspended polymer particles have an average particle size in the range from 150 to 450 nm, the suspension includes a proportion of sugar or sugar alcohol dissolved therein in the range from 25 to 250 g/l and the suspension has a pH in the range from 8 to 10.

The present invention relates to a diagnostic reagent for quantitative determination of procalcitonin in a sample, wherein the reagent is an aqueous suspension of polymer particles with antibodies against procalcitonin covalently bound to said polymer particles. The present invention also relates to a method for preparing such a diagnostic reagent.

Procalcitonin (PCT) is a precursor of the hormone calcitonin and also one of the most important markers in the diagnosis of sepsis. PCT is standardly used, in particular for post-operative control of sepsis. The reason for this is that liver and fat cells produce more PCT in cases of in particular bacterial infections after operations. In daily clinical routine, however, PCT is not only used post-operatively, but also generally as an accompanying measure for the initial diagnosis and differentiation of sepsis caused by viruses, bacteria, fungi and protozoa/parasites and then for follow-up control, i.e., to check the effectiveness of the medication used.

Sepsis has several ranges that are diagnostically relevant. Below 0.5 ng/ml sepsis is unlikely. Above 0.5-2 ng/ml inflammation is likely. The presence of sepsis has to be clarified by repeated time-delayed measurement of PCT and, if necessary, other parameters. Above 2-10 ng/ml sepsis with probable bacterial association is present. Above 10 ng/ml, severe septic shock is present. This classification is used to characterise sepsis and the measures to be derived from said characterisation. This is essential especially with regard to the time factor.

Every hour in which sepsis is not diagnosed or a diagnosis that is delayed by one hour statistically increases mortality by 7%. For this reason, sepsis is the third leading cause of death in all regions of the world. With this in mind, extremely high demands with regard to sensitivity and reproducibility have to be placed on the diagnosis of sepsis. Even after prolonged periods of storage, the reagents used for the diagnosis have to provide reliable results.

A variety of diagnostic methods based on immunoassays, which can be used to quantitatively determine PCT in a patient's blood, are known from the prior art. Antibodies against PCT are bound to polymer particles and then, after reaction with the PCT contained in the sample to be examined, analysis can be carried out.

The CLIA method (chemiluminescence immunoassay) requires the use of a chemiluminescence reagent component, so that the analysis can be carried out on the basis of the occurring chemiluminescence. A special device is needed for this, in which the particles have to generally be continuously resuspended.

In the PETIA method (particle-enhanced turbidimetric immunoassay), a turbidimetric analysis is carried out on the basis of the decrease in the transmission of light through the reaction fluid. The more PCT in a sample, the stronger the crosslinks that occur between the particles occupied with antibodies, which results in greater turbidity of the liquid and thus to a decrease in transmission. There is consequently no need for an additional chemiluminescence reagent, and the measurement can be carried out with a simple photometer system as is available in practically every diagnostic laboratory.

For the accuracy and diagnostic relevance of the turbidimetric analysis, it is absolutely critical that the degree of turbidity that occurs as a result of the reaction with PCT always reliably correlates with the quantitative amount of PCT in the sample. This is not the case, for example, if the particles loaded with antibodies spontaneously/non-specifically agglutinate or sediment or if their specific antigen-binding capacity directed against PCT changes over time after preparation.

There is therefore a need for a diagnostic reagent that is suitable for turbidimetric analysis with a simple photometer and has high sensitivity for quantitative determination of procalcitonin in a sample, wherein the reagent is an aqueous suspension of polymer particles with antibodies against procalcitonin covalently bound to said polymer particles, in which no or only an extremely slight tendency to agglutination/sedimentation is detectable even after longer standing times and the specific reactivity of the particles remains largely unchanged.

This object is achieved according to the invention in that
the suspended polymer particles have an average particle size in the range from 150 to 450 nm,
the suspension comprises a proportion of sugar or sugar alcohol dissolved therein in the range from 25 to 250 g/l and
the suspension has a pH in the range from 8 to 10.

The combination of features proposed according to the invention provides a diagnostic reagent for the quantitative determination of PCT that has a very high sensitivity in the here relevant range of extremely low PCT concentrations in a sample. This high sensitivity is moreover maintained even during prolonged storage for up to 24 months. This high storage stability is noteworthy in particular in view of the fact that the polymer particles used according to the invention have a relatively large particle size of 150 nm and more.

The large particle size of the polymer particles is expressly desired in particular with regard to the required high sensitivity of the diagnostic reagent. The relatively large particles have a correspondingly large surface area, so that a correspondingly large number of antibodies against PCT can be covalently bound thereon. There are therefore more potential coupling sites for the PCT molecules, which leads to an increased sensitivity of the diagnostic reagent.

The large particle size is also advantageous in terms of the light scattering properties. It has been found that the “light yield” is significantly better with the polymer particles according to the invention. With the same degree of crosslinking, the particles according to the invention essentially reamplify the signal. The larger the particles, the stronger the signal. The underlying principles are effects such as Rayleigh scattering, Mie scattering, etc. on colloidal nanoparticles in suspension.

With the present invention it was surprisingly possible to achieve better storage stability than would have been expected despite the large particle size. Larger particles typically have a greater tendency to sediment and are therefore more difficult to resuspend after a longer storage period. However, the combination proposed according to the invention of sugar or sugar alcohol dissolved in the suspension and the pH to be set in the suspension surprisingly makes it possible to obtain a highly storage-stable suspension with consistent reactivity even with the relatively large particles of the invention.

The diagnostic reagent claimed here is for the quantitative determination of PCT in a sample. In the context of the present invention, a “sample” is understood to be any material prepared for the purpose of analysis that contains an amount of PCT to be analysed. In most cases, the sample will be a sample of fresh whole blood that has possibly been appropriately prepared for the purpose of performing the analysis. However, the present invention also encompasses other liquid samples containing PCT, such as standard solutions and calibrators.

The diagnostic reagent of the present invention is suitable for quantitative PCT determination. “Quantitative determination” in this context means that inferences can be made about the amount of PCT in the sample from the amount of PCT molecules bound to the polymer particles.

In the reagent according to the invention, the polymer particles are present in an aqueous suspension. In this context, the term “aqueous suspension” refers to a slurry of the polymer particles loaded with antibodies against procalcitonin in water or an aqueous solution in which sugar, sugar alcohol and/or buffer substances are dissolved. In the context of the present invention, the term “suspension” must be interpreted narrowly and means that almost all of the particles are suspended in the aqueous phase and are not sedimented. A suspension in the sense of the present invention is therefore present when at least 95%, at least 96%, at least 97%, at least 98% or even at least 99% of the polymer particles are freely suspended.

According to the invention, the average particle size of the polymer particles ranges from 150 to 450 nm. In certain embodiments within the claimed range, the average particle size is preferably >190 nm, >240 nm, or even >300 nm. In other embodiments within the claimed range, the average particle size is preferably <410 nm, <360 nm, or even <300 nm. In the case of the present invention, the particle size of the polymer particles is determined by means of dynamic light scattering at 25° C., e.g., using Malvern's Zetasizer Pro. The average particle size refers to the numerical average.

The polymer material of which the polymer particles consist is preferably selected from acrylic polymer, dextran-epichlorohydrin copolymer, polymethyl methacrylate, polystyrene, silica (silica gel), and combinations thereof. In certain embodiments, the polymer particles consist entirely of one polymer material or mixtures thereof. In other embodiments, the particles consist of multiple layers of different polymers.

In specific embodiments, the particles can comprise a core and one or more layers coated on to that core. The core and one or more layers coated on to it can be comprised of one polymer material, different polymer materials or a non-polymeric material, provided that the polymer particles consist predominately of polymer material.

A polymer particle in the sense of the present invention, is present when the particles consist of at least 80 wt %, at least 90 wt %, at least 95 wt % or 100 wt % of a polymer material or a combination of different polymer materials.

The overall density of the polymer particle is preferably in the range from 0.9 to 1.1 g/cm³, and particularly preferably in the range from 1.0±0.5 g/cm³.

On the surface, the polymer particles preferably comprise functional groups, via which covalent binding of the antibodies to the surface of the polymer particles can take place. These functional groups are preferably selected from a carboxyl group (—COOH), primary amine group (—RNH₂), aromatic amine group (—ArNH₂), chloromethyl group (—CH₂CI), aromatic chloromethyl group (—ArCH₂Cl), amide group (—CONH₂), hydrazide group (—CONHNH₂), aldehyde group (—CHO), hydroxyl group (—OH), thiol group (—SH), epoxy group and biotin-avidin.

The aqueous portion of the suspension of the diagnostic reagent according to the invention contains a dissolved sugar or sugar alcohol portion with a concentration in the range from 25 to 250 g/l. In certain embodiments, the concentration of sugar and/or sugar alcohol is in the range from 50 to 200 g/l. In some embodiments, the concentration is 50 to 150 g/l, in other embodiments the concentration is 150 to 250 g/l.

In certain embodiments, the sugar or sugar alcohol dissolved in the aqueous portion of the suspension is selected from sucrose, mannitol, sorbitol, xylitol, maltitol, raffinose, rhamnose, and combinations thereof. Where a combination of sugars/sugar alcohols is used, the above amounts refer to the sum of the proportions of sugars/sugar alcohols of that combination.

The pH of the aqueous suspension, in which the polymer particles loaded with antibodies are suspended, is in the range from 8 to 10. In some embodiments the pH is in the range from 9.0 to 10.0. In certain embodiments, the pH of the suspension is >9. In some embodiments the pH of the suspension is in the range from 9.1 to 10.0. In certain embodiments the pH of the suspension is 9.0±0.5. In special embodiments, the pH of the suspension is 9.5±0.1.

The diagnostic reagent claimed according to the invention is characterised by a very high storage stability. Among other things, this is expressed in that the turbidity of the suspension is stable compared to the initial value even after several months. The suspension of the present invention is in particular characterised in that, within 90, 120, 150 or even 180 days from the time of preparation of the suspension, the absorbance of the suspension at 660 nm deviates by less than 5% from the initial value on day zero. In certain embodiments, the deviation is less than 5% even within 12 months or even within 24 months. In certain embodiments, the deviation is even less than 3% or even less than 2%. In certain embodiments of the invention, the slight deviation in the degree of turbidity within the aforementioned time periods can also be measured at other wavelengths in the range from 340 to 800 nm.

In a particular embodiment of the present invention, the antibodies which are covalently bound to the polymer particles are monoclonal antibodies against PCT. In other embodiments, the antibodies are polyclonal antibodies against PCT. In yet other embodiments of the invention, the polymer particles are loaded with covalently bound recombinant antibodies against PCT or covalently bound antibody fragments against PCT.

The present invention also claims a method for preparing a diagnostic reagent of the type described above. In this method, the polymer particles are first brought into contact with the antibodies against PCT, whereby this takes place under conditions in which a covalent binding of the antibodies to the surface of the polymer particles occurs. The loading of the polymer particles with the antibodies takes place in an aqueous suspension over a specific period of time at a specific temperature and a specific pH.

The method according to the invention is characterised in that the covalent binding of the antibodies via the functional groups on the surface of the polymer particles takes place at a pH of 3 to 6. In certain embodiments, the covalent binding takes place at a pH from 3 to 5. In certain embodiments, the pH in the reaction leading to the covalent binding of the antibodies via the functional groups on the surface of the polymer particles is 4.0±0.5.

The pH in the reaction leading to the covalent binding of the antibodies via the functional groups on the surface of the polymer particles is preferably set using inorganic or organic buffer substances, such as borate, citrate, phosphate, malate, maleate, succinate, acetic acid/acetate. The pH is preferably set/adjusted using hydrochloric acid/sodium hydroxide solution.

In certain embodiments of the method according to the invention, the covalent binding of the antibodies via the functional groups on the surface of the polymer particles takes place at a temperature in the range from 20 to 29° C. In other embodiments, this reaction takes place in the range from 30 to 34° C., or even in the range from 35 to 45° C.

In certain embodiments of the method according to the invention, the covalent binding of the antibodies takes place in one of the aforementioned defined pH ranges and at a temperature in one of the above defined ranges over a period of 20 to 80 hours. In certain embodiments the reaction takes place over a period of >30, >40, >50 or even >60 hours.

At the end of the time periods specified above, the pH of the suspension of the polymer particles now loaded with antibodies is lowered to the range of 8 to 10. For this purpose, one or more of the abovementioned alkalising agents or buffer substances can be added.

The pH in the suspension is preferably set using buffer substances comprising amine groups, such as EPPS, HEPPS, tricine, tris, glycylglycine, bicine, TAPS, boric acid, ethanolamine, CHES, glycine and CAPS. The pH is preferably set/adjusted using hydrochloric acid/sodium hydroxide solution.

The polymer particles obtained using the method according to the invention, which are loaded with antibodies or antibody fragments, exhibit extremely high sensitivity and very high storage stability, as previously described.

For the purpose of the original disclosure, it should be noted that all of the features as they become apparent to a person skilled in the art from the present description, the figures and the claims, even if they have been specifically described only in connection with specific other features, can be combined both individually and in any combination with other features or groups of features disclosed here, insofar as this has not been expressly excluded or technical circumstances make such combinations impossible or pointless. A comprehensive, explicit presentation of all conceivable combinations of features is omitted here solely for the sake of brevity and legibility of the description.

It should further be noted that it is obvious to the person skilled in the art that the following examples for embodiments are intended merely to illustrate examples of possible embodiments of the present invention. The person skilled in the art will therefore moreover readily understand that all other embodiments having the features or combinations of features according to the invention mentioned in the claims are also within the scope of protection of the invention. A comprehensive, explicit presentation of all conceivable embodiments is omitted here solely for the sake of brevity and legibility of the description.

### FIGURES

FIG. 1: Results of the examination of the effect of incubation time on sensitivity

FIG. 2: Results of the examination of the effect of pH on sensitivity

FIG. 3: Results of the examination of the storage stability

FIG. 4: Results of the examination of the storage stability

FIG. 5: Results of the examination of the effect of particle size on the measurement precision

FIG. 6: Results of the examination of the effect of particle size on sensitivity and linearity

FIG. 7: Results of the examination of the effect of the sugar concentration on calibration

FIG. 8: Results of the examination of the effect of different combinations of sucrose and pH on sensitivity

FIG. 9: Results of the examination of the effect of different combinations of sucrose and pH on sensitivity

FIG. 10: Results of the examination of the effect of different combinations of sucrose and pH on sensitivity

FIG. 11: Results of the examination of the effect of incubation time on sensitivity

FIG. 12 Results of the examination of the stability of reactivity at a pH of 8.1

FIG. 13 Results of the examination of the stability of reactivity at a pH of 9.0

FIG. 14 Results of the examination of the stability of reactivity at a pH of 9.5

FIG. 15 Results of the examination of long-term stability

FIG. 16 Results of the examination of long-term stability

### EXAMPLES FOR EMBODIMENTS

In the following, a variety of comparative tests are presented, in which different parameters of the method for producing the diagnostic reagent according to the invention are changed. For this purpose, polymer particles of polystyrene were loaded with antibodies against PCT under different conditions.

In one embodiment variant, the polymer particles comprised carboxyl groups (—COOH) as the functional group and, in another embodiment variant, the polymer particles comprised chloromethyl groups (—CH₂Cl) as the functional group.

### I. Embodiment Variant with Chloromethyl Groups

### 1. Incubation Time

The polymer particles used had an average particle size of >350 nm. The particles were coupled to the antibodies over a period of 15 to 48 hours, and the results of the different batches are shown in FIG. 1.

These results are expressed in terms of absorbance at 660 nm, and the comparatively high absorbance values of the batch in which the incubation time was 48 hours demonstrate that the batches incubated for a longer period of time provide significantly higher sensitivity at the respective concentration of PCT in the sample, because clearly higher proportions of the PCT contained in the sample are bound than in the other batches. The amount of PCT contained in the sample can thus be determined in a quantitatively more accurate manner.

2. pH

In these experiments, the pH during the coupling reaction was varied in the range from 4 to 9.

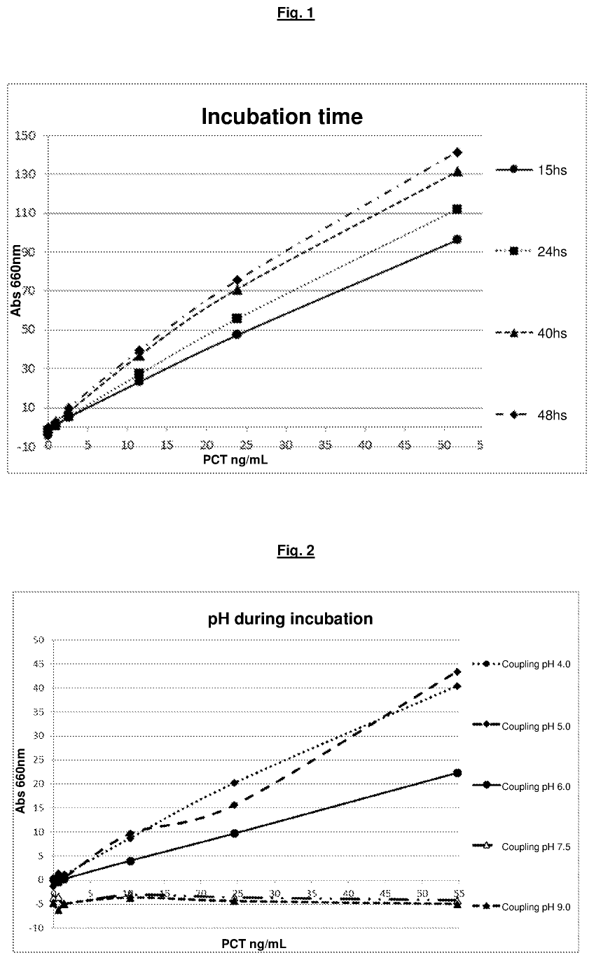

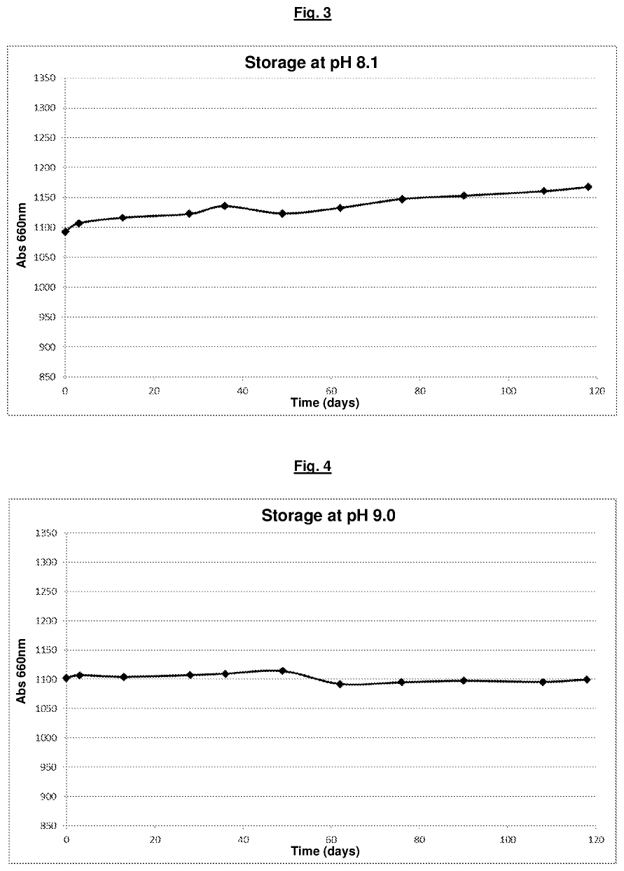

The results shown in FIG. 2 demonstrate that better loading efficiency is achieved at lower pH values. In this way, polymer particles are obtained which have a higher binding capacity to PCT and thus a higher sensitivity.

### 3. Storage Stability

To check the suspension stability of the diagnostic reagent according to the invention, an aqueous suspension of the polymer particles was mixed with a teaching sample (physiological saline solution) and the turbidity of the resulting mixture was then determined. The thus used polymer particles were stored under different pH conditions for a period of up to 120 days.

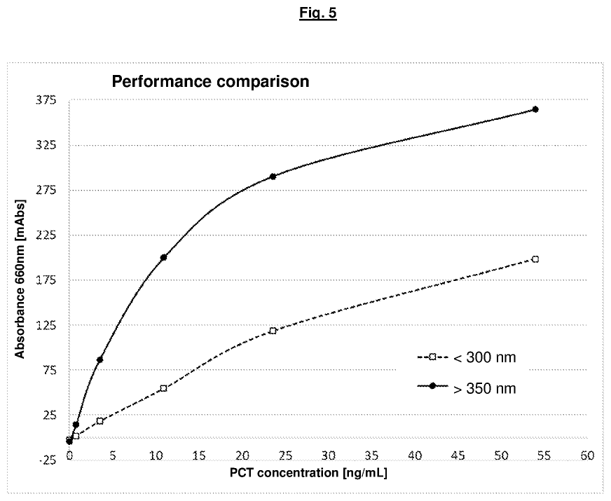

The results shown in FIGS. 3 and 4 demonstrate that there was significantly less turbidity when the polymer particles had been stored at a pH of 9.0 than in the samples stored at a pH of 8.1, in particular for the samples stored for 60 days and more. This clearly demonstrates that the tendency to agglutination and sedimentation is significantly lower in the batches stored at pH 9.

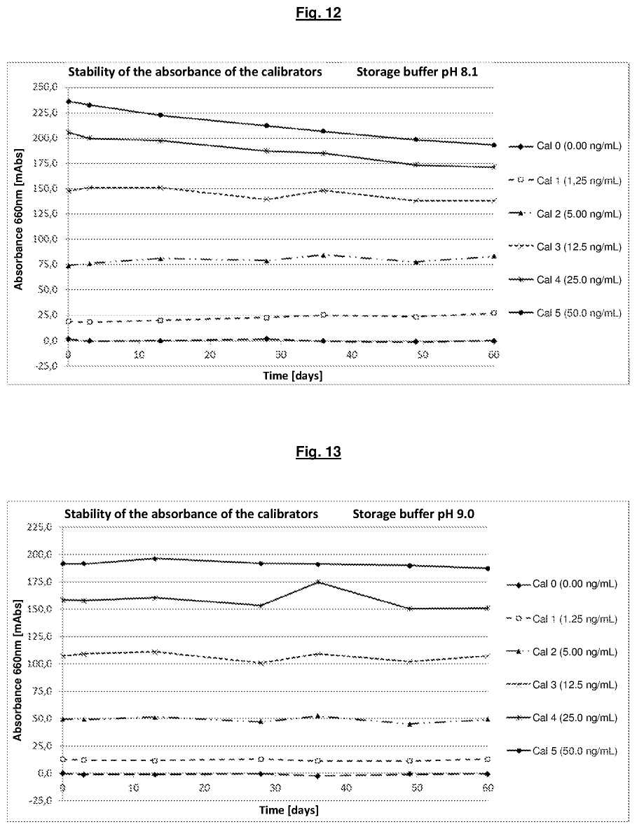

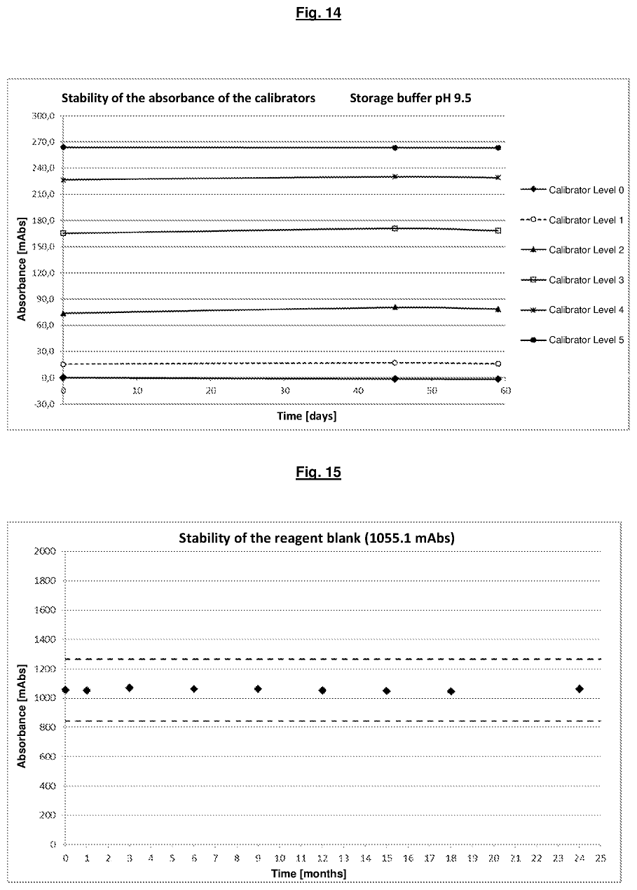

As can be seen in FIGS. 12 to 14, the stability of reactivity in the higher pH range, namely in the range from pH 9.0 (see FIG. 13) to pH 9.5 (see FIG. 14), is better than in the lower range (see FIG. 12: pH 8.1). In the period of 60 days studied here, a particularly high stability of reactivity can be observed at a pH in the range >9 (see FIG. 14: pH 9.5).

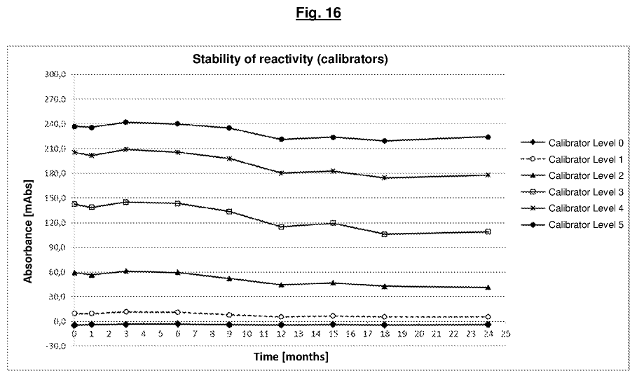

FIGS. 15 and 16 demonstrate the high long-term stability of up to 24 months in an embodiment with a storage buffer having a pH of 9.0. The small jump in the curve at 12 months in the results of the examination of the reactivity of the calibrators can be explained by the lamp on the detector being changed after 12 months.

### II. Embodiment Variants with Different Particle Sizes

To investigate the effect of particle size, polymer particles comprising chloromethyl groups and having an average particle size of >350 nm were compared with polymer particles comprising chloromethyl groups and having an average particle size of <300 nm.

FIG. 5 shows that, especially in the range of concentrations 0.2-2 ng/mL, in which the two medically relevant decision ranges of procalcitonin are located, the reactivity is much stronger for the polymer particles having an average particle size of >350 nm than for the polymer particles having an average particle size of <300 nm. This significantly improves the measurement precision.

The recovery (accuracy of concentration) is also better with the polymer particles having an average particle size of >350 nm than with the polymer particles having an average particle size of <300 nm (see Table 1 below).

TABLE 1

| | Target | | | |
|---|---|---|---|---|
| | value PCT | Range | Recovery | Recovery |
| | [ng/mL] | [ng/mL] | <300 nm | >350 nm |
| Control Level 1 | 0.80 | 0.60-1.00 | 1.14-1.12 | 0.75-0.74 |
| Control Level 2 | 10.85 | 8.68-13.03 | 10.58-10.57 | 10.46-10.24 |

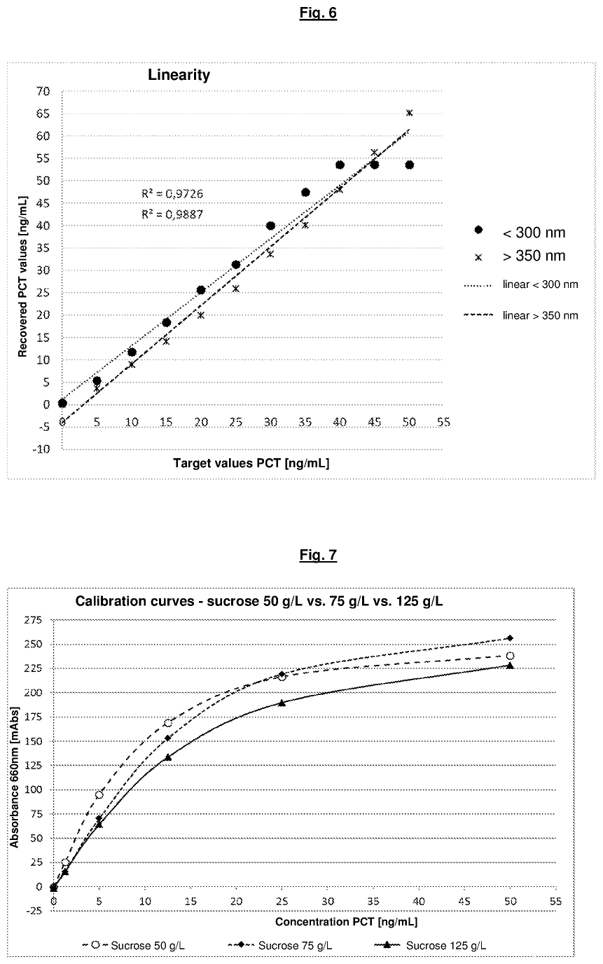

FIG. 6 shows the results of the measurement of the sensitivity (measured as precision at the cut-off) and the linearity of the recovery of a diluted sample.

With the polymer particles having an average particle size of <300 nm, linear measurement is possible only to the target value 35 ng/mL. Higher concentrations are no longer differentiated.

The polymer particles having an average particle size of >350 nm have linear recovery up to 65 ng/mL. The correlation coefficient of the larger particles is accordingly also higher than that of the smaller particles.

The recovery and precision of a sample at the medically relevant cut-off (0.5 ng/mL PCT) are also significantly better for the larger particles than for the smaller ones (see Table 2 below).

TABLE 2

Measured concentration PCT [ng/mL]

| | <300 nm | >350 nm |
|---|---|---|
| Mean [ng/mL] (target | 1.01 | 0.60 |
| value = 0.5 ng/mL) | | |
| Std. deviation | 0.0773 | 0.0294 |
| CV % | 7.65 | 4.86 |

### III. Embodiment Variants with Different Sugar Concentrations

To investigate the effect of different sugar concentrations, polymer particles comprising chloromethyl groups and having an average particle size of >350 nm were analysed in storage buffers having different sugar concentrations.

FIG. 7 shows that a comparison of 50 g/L vs. 75 g/L vs. 125 g/L sucrose in the storage buffer yields similar calibration curves. However, the precision (measured as CV % of 20-fold determination of a sample containing 0.5 ng/mL PCT) becomes significantly better (CV % lower) the higher the sucrose concentration (see Table 3 below).

TABLE 3

| | Sucrose | Sucrose | Sucrose |
|---|---|---|---|
| | 50 g/L | 75 g/L | 125 g/L |
| Precision [CV %] | 13.97 | 12.53 | 6.53 |

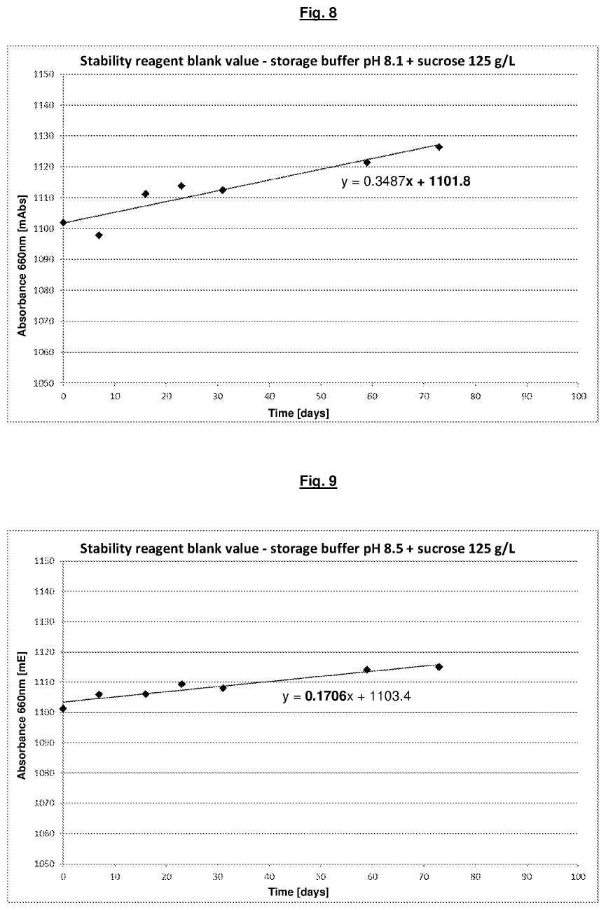

FIGS. 8, 9 and 10 show data of the combined effect of sucrose+pH. The chloromethyl particles are resuspended in three storage buffers with sucrose 125 g/L and pH 8.1/8.5 and 9.0. The slope of the linear regression line of the reagent blank values on the different measurement days corresponds to the aggregation of the particles and should be as close to 0 as possible (Excel slide “Combined effect suc-pH”).

### IV. Embodiment Variant with Carboxyl Groups

The polymer particles used had an average particle size of <310 nm. The particles were coupled to the antibodies over a period of 12 to 24 hours and the pH in the coupling reaction was 4 to 6.

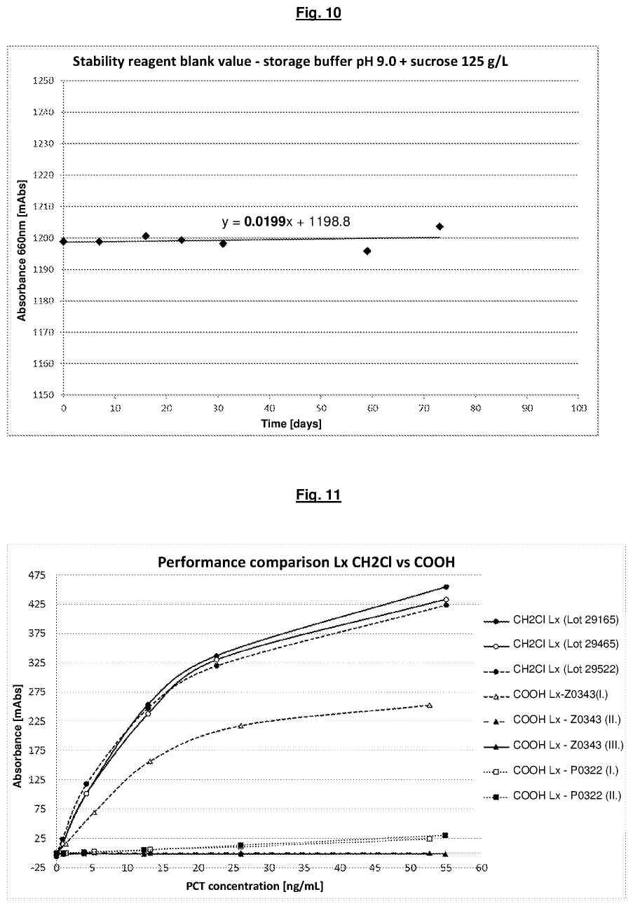

As the results shown in FIG. 11 indicate, good reactivity can be achieved with polymer particles comprising a carboxyl group. By comparison, however, the polymer particles comprising chloromethyl groups exhibit a significantly stronger reactivity.

Polymer particles comprising a carboxyl group also have to be activated (EDC+NHS) prior to the coupling reaction in order to make the COOH groups reactive. This activation is complex and does not always work in a reproducible and batch-homogeneous manner. In the present case, only one activation out of five attempts was actually successful. By comparison, polymer particles comprising chloromethyl groups couple very reliably (here the data from three batches).

1. A diagnostic reagent for quantitative determination of procalcitonin in a sample, wherein the reagent is an aqueous suspension of polymer particles with antibodies against procalcitonin covalently bound to said polymer particles, characterised in that
the suspended polymer particles have an average particle size in the range from >300 nm to 450 nm,
the suspension comprises a proportion of sugar or sugar alcohol dissolved therein in the range from 25 to 250 g/l, and
the suspension has a pH in the range from 8.5 to 10.

2. The diagnostic reagent according to claim 1, wherein the polymer of the polymer particles is selected from acrylic polymer, dextran-epichlorohydrin copolymer, polymethyl methacrylate, polystyrene and silica.

3. The diagnostic reagent according to claim 1, wherein that the covalent bond of the antibodies is formed via functional groups disposed on the surface of the polymer particles, wherein the functional groups are selected from a carboxyl group (—COOH), primary amine group (—RNH₂), aromatic amine group (—ArNH₂), chloromethyl group (—CH₂CI), an aromatic chloromethyl group (—ArCH₂CI), amide group (—CONH₂), hydrazide group (—CONHNH₂), aldehyde group (—CHO), hydroxyl group (—OH), thiol group (—SH), epoxy group and biotin-avidin.

4. The diagnostic reagent according to claim 1, wherein the sugar or sugar alcohol is selected from sucrose, mannitol, sorbitol, xylitol, maltitol, raffinose, rhamnose, and combinations thereof.

5. The diagnostic reagent according to claim 1, wherein the absorbance of the suspension at 660 nm within 90, 120, 150 or 180 days or even within 12 months or 24 months from the time of preparation of the suspension deviates by less than 5%, less than 3% or even less than 2% from the initial value on day 0.

6. The diagnostic reagent according to claim 1, wherein the antibodies against procalcitonin covalently bound to the polymer particles are monoclonal polyclonal or recombinant antibodies or antibody fragments.

7. The diagnostic reagent according to claim 1, wherein the suspension has a pH in the range from 9.0 to 10.0.

8. A method for preparing the diagnostic reagent according to claim 1, comprising covalent binding the antibodies via the functional groups on the surface of the polymer particles at a pH of 3 to 6.

9. The method according to claim 8, wherein the covalent binding of the antibodies via the functional groups on the surface of the polymer particles takes place at a temperature in the range from 20 to 29° C.

10. The method according to claim 8, wherein the covalent binding of the antibodies takes place in a defined pH range and at a defined temperature over a period of 20 to 80 hours.

11. Diagnostic reagent for quantitative determination of procalcitonin in a sample, wherein it is obtained using the method according to claim 8.
